(19) United States
(12) Patent Application Publication
(10) Pub. No.: US 2024/0065986 A1
(43) Pub. Date: Feb. 29, 2024

(54) OPTIMIZED INGREDIENT MATRICES FOR ENHANCED HYDRATION AND MUSCLE RECOVERY PRE-, INTRA-, AND POST-EXERCISE

(71) Applicant: GAMMA DELTA IP, INC., Houston, TX (US)

(72) Inventors: Jason Kelly Allison, Houston, TX (US); Guillermo A. Amtmann, Houston, TX (US); John Burton Steele, Houston, TX (US); Nanda G. Karri, Houston, TX (US)

(21) Appl. No.: 17/900,405

(22) Filed: Aug. 31, 2022

(51) Int. Cl. A61K 31/05; A23L 2/60; A23L 33/105; A23L 33/125; A23L 33/16; A23L 33/175; A61K 9/00; A61K 47/02; A61K 47/12; A61K 47/26

(52) U.S. Cl. CPC A61K 31/05; A23L 2/60; A23L 33/105; A23L 33/125; A23L 33/16; A23L 33/175; A61K 9/0095; A61K 47/02; A61K 47/12; A61K 47/26; A23V 2002/00

(57) ABSTRACT

A hydration formula developed through evidence-based research to keep people hydrated for peak performance. Ingredients used to deliver key components include calcium lactate, dextrose, a branched-chain amino acid, a citric acid, a citrate, stevia, potassium sorbate, sodium benzoate, and water. The synergistic action of the disclosed formulation allows for rapid hydration or rehydration of the human body especially after physical activity and exercise, and in particular after high intensity exercise or activity.

### FIELD

The present disclosure generally relates to hydration formula developed through evidence-based research to keep people hydrated for peak performance.

### BACKGROUND

Drinking fluids is critical for people to stay healthy and maintain optimal functioning of various parts of the body, including your heart, brain, and muscles. Fluids can help nourish cells in the body and flush bacteria from the system.

Dehydration can become a serious condition that can lead to health problems. Keeping the body hydrated helps muscles work efficiently and prevent other adverse conditions ranging from headache to heat stroke.

Electrolytes are also vital for physiological functions such as thermoregulation, nerve impulse conduction, muscle contraction, cardiovascular function and fluid homeostasis.

Additionally, the body requires certain electrolytes such as magnesium and calcium for muscle proteins and enzymes to fold and function properly. During rigorous physical exercise, water loss and sweating results in net losses or deficits in these important electrolytes, impairing performance. Proper preparative and restorative electrolyte supplementation is critical for optimum performance and physiological function.

The present disclosure relates to a hydration formula developed through evidence-based research to keep people hydrated for peak performance.

### DETAILED DESCRIPTION OF THE EMBODIMENTS

Before explaining the present disclosure in detail, it is to be understood that the disclosure is not limited to the specifics of particular embodiments as described and that it can be practiced, constructed, or carried out in various ways.

While embodiments of the disclosure have been shown and described, modifications thereof can be made by one skilled in the art without departing from the spirit and teachings of the disclosure. The embodiments described herein are exemplary only, and are not intended to be limiting.

Specific structural and functional details disclosed herein are not to be interpreted as limiting, but merely as a basis of the claims and as a representative basis for teaching persons having ordinary skill in the art to variously employ the present embodiments. Many variations and modifications of embodiments disclosed herein are possible and are within the scope of the present disclosure.

Where numerical ranges or limitations are expressly stated, such express ranges or limitations should be understood to include iterative ranges or limitations of like magnitude falling within the expressly stated ranges or limitations.

The use of the word “a” or “an” when used in conjunction with the term “comprising” in the specification may mean “one,” but it is also consistent with the meaning of “one or more,” “at least one,” and “one or more than one.”

The word “about”, when referring to values, means plus or minus 5% of the stated number.

The use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

When methods are disclosed or discussed, the order of the steps is not intended to be limiting, but merely exemplary unless otherwise stated.

Accordingly, the scope of protection is not limited by the description herein, but is only limited by the claims which follow, encompassing all equivalents of the subject matter of the claims. Each and every claim is hereby incorporated into the specification as an embodiment of the present disclosure. Thus, the claims are a further description and are an addition to the embodiments of the present disclosure.

The inclusion or discussion of a reference is not an admission that it is prior art to the present disclosure, especially any reference that may have a publication date after the priority date of this application. The disclosures of all patents, patent applications, and publications cited herein are hereby incorporated by reference, to the extent they provide background knowledge; or exemplary, procedural or other details supplementary to those set forth herein.

The embodiments of the present disclosure generally relate to a hydration formula developed through evidence-based research to keep people hydrated for peak performance.

All values are given based upon sixteen fluid ounces of the formulation. However, the ratios can be held as disclosed and the amounts adjusted for different volumes of the formulation.

Proper hydration for peak performance requires not only water, but a number of other components to help the body to operate and recover optimally. In addition, it is desirable to have a pleasant tasting formulation to encourage consumption.

Key components to be included within the formulation are electrolytes, branched-chain amino acids, lactate, and dextrose. In embodiments, cannabidiol can be added for additional benefits.

Electrolytes are vital for physiological functions such as thermoregulation, nerve impulse conduction, muscle contraction, cardiovascular function and fluid homeostasis. Additionally, the body requires certain electrolytes such as magnesium and calcium for muscle proteins and enzymes to fold and function properly. During rigorous physical exercise, water loss, and sweating results in net losses or deficits in these important electrolytes, thereby impairing performance. Proper preparative and restorative electrolyte supplementation is critical for optimum performance and physiological function.

Exemplary electrolytes required for optimal performance include Sodium (Nat), Potassium (K⁺), Calcium (Ca²⁺), Magnesium (Mg²⁺), and Chloride (Cl⁻) ions.

BCAAs are important building blocks for muscle proteins and have been shown to be important during strenuous exercise. BCAAs consist of leucine, valine, and isoleucine, which contain branched, aliphatic side chains and are among the nine essential amino acids. They account for −35% of the essential amino acids in muscle proteins.

Aside from their importance in muscle protein building and maintenance, BCAAs can be oxidized in muscle tissue during strenuous exercise creating an expanded intermediate pool for the citric-acid cycle yielding an increased amount of ATP (energy source of the human body). Importantly, this alternative to glycolysis for energy production under anaerobic (no oxygen) conditions limits the amount of lactic acid production, which subsequently increases one's lactic acid threshold allowing for peak, sustained performance.

Interestingly, experiments have shown that glucose plus BCAA supplementation results in a marked increase in plasma concentrations of BCAAs. This is likely a result of the increased solubility of BCAAs in the presence of glucose (dextrose). This synergistic effect is capitalized upon by the formulation of the present disclosure to yield optimal performance.

Lactate, once widely believed to cause muscle fatigue and cramping associated with intense exercise, is now known to relieve these very symptoms. Hydrogen ion buildup secondary to ATP breakdown is now suspected to be the real cause of fatigue and cramping.

Lactate improves tolerance of this hydrogen ion accumulation (low pH, acidosis). Lactate supplementation not only directly increases lactate levels but also increases the liver's ability to turn over lactate, a marker for faster recovery. Indeed, precisely because large lactate loads, such as those generated by intense training, improve liver turnover, lactate supplementation is now being considered a possible exercise mimetic. Furthermore, lactate itself serves as an alternative cellular energy source during intense workouts

Dextrose is the dextrorotary (isomer) form of glucose. It is a simple sugar that is rapidly absorbed and converted to glycogen for storage or adenosine triphosphate (ATP), the energy source for our bodies. The rapid absorption of dextrose creates an osmotic gradient that pulls water with it leading to quicker hydration. Research indicates that a hydration drink with a dextrose concentration of less than six percent weight by volume supplemented with sodium supports optimal oral rehydration. The present disclosure also incorporates this synergy.

Cannabidiol (CBD) is the second most prevalent active ingredient of cannabis (marijuana). CBD can be directly derived from the hemp plant, which is a relative of marijuana.

CBD is commonly used to address anxiety, and for patients who suffer from insomnia. In addition, CBD has been used to treat seizure disorders.

Recent data indicates that CBD is effective for treating pain and inflammation. By combining CBD with the ingredients above, it is contemplated that muscle fatigue is reduced and recovery enhanced, in addition to providing mental assistance and having a calming effect.

Ingredients used to deliver the above components include calcium lactate, dextrose, a branched-chain amino acid, a citric acid, a citrate, stevia, potassium sorbate, sodium benzoate, and water. In embodiments, the citrate can be a sodium citrate, a potassium citrate, a magnesium citrate, or combinations thereof.

In embodiments, the formulation comprises about 800 to about 1,200 milligrams of calcium lactate, about 950 to about 28,000 milligrams of dextrose, about 2,000 to about 5,000 milligrams of branched-chain amino acids, about 1,400 to about 1,900 milligrams of citric acid, about 150 to about 1,200 milligrams of citrate, and about 270 milligrams of stevia. The formulation can also comprise about 142 milligrams of potassium sorbate, or about 142 milligrams of sodium benzoate. Further, the formulation can comprise a citrate, such as about 600 to about 1,200 milligrams of sodium citrate, about 350 to about 750 milligrams of potassium citrate, about 150 to about 550 milligrams of magnesium citrate, or combinations thereof.

In embodiments, the formulation comprises about 1000 milligrams of calcium lactate, about 10,000 milligrams of dextrose, about 4,000 milligrams of branched-chain amino acids, about 1700 milligrams of citric acid, and about 270 milligrams of stevia. The formulation can also comprise about 142 milligrams of potassium sorbate, or about 142 milligrams of sodium benzoate. Further, the formulation can comprise a citrate, such as about 800 milligrams of sodium citrate, about 450 milligrams of potassium citrate, about 350 milligrams of magnesium citrate, or combinations thereof.

In embodiments, 15 to 30 milligrams of cannabidiol can be added to the formulation.

The combination of electrolytes, branched-chain amino acids, lactate, and dextrose, in the disclosed ratios provides an optimum blend of ingredients to hydrate the human body. By combining the formulation with cannabidiol, pain relieving and inflammation reducing characteristics are added to the formulation.

The synergistic action of the disclosed formulation allows for rapid hydration or rehydration of the human body especially after physical activity and exercise, and in particular after high intensity exercise or activity. High intensity exercises are defined as those performed to maintain heart rates at above 80% of maximum heart rate.

Often the performance of high intensity exercises or activities result in delayed onset muscle soreness (DOMS), excessive lactic acid buildup, and rapid dehydration. Often inflammation is experienced in muscle tissue.

The present disclosure aids in recovery as well as prevention of some of the issues discussed above. Buffering of intra and extra-cellular fluids with the use of citrates aids in reducing lactic acid buildup in muscles. The combination of sugars and branched-chain amino acids aids in muscle recovery and muscle building following exercise. Citrates and the various salts utilized further aid in rehydration.

The combination of the disclosed ingredients aid in rehydration, preventing DOMS, as well as acting in an anti-inflammatory capacity. All of these results promote effective immune cell function and muscle health.

While the present disclosure emphasizes the presented embodiments, it should be understood that within the scope of the appended claims, the disclosure might be embodied other than as specifically enabled herein.

What is claimed is:

1. A hydrating beverage formulation comprising:
a. calcium lactate;
b. dextrose;
c. a branched-chain amino acid;
d. a citric acid;
e. a citrate;
f. stevia; and
g. water.

2. The formulation of claim 1, further comprising:
a. potassium sorbate; or
b. sodium benzoate;

3. The formulation of claim 1, wherein the citrate is a:
a. a sodium citrate;
b. a potassium citrate; or
c. a magnesium citrate.

4. The formulation of claim 1, further comprising cannabidiol.

5. The formulation of claim 1, wherein the branched-chain amino acid is leucine, isoleucine, or valine.

6. The formulation of claim 1, wherein the water is distilled water, deionized water, buffered water, filtered water, or combinations thereof.

7. The formulation of claim 1, wherein the formulation comprises per sixteen fluid ounces:
a. about 800 to about 1,200 milligrams of calcium lactate;
b. about 950 to about 28,000 milligrams of dextrose;
c. about 2,000 to about 5,000 milligrams of branched-chain amino acids;
d. about 1,400 to about 1,900 milligrams of citric acid;
e. about 150 to about 1,200 milligrams of citrate; and
f. about 270 milligrams of stevia.

8. The formulation of claim 1, wherein the formulation comprises per sixteen fluid ounces:
a. about 1000 milligrams of calcium lactate;
b. about 10,000 milligrams of dextrose;
c. about 4,000 milligrams of branched-chain amino acids;
d. about 1,700 milligrams of citric acid; and
e. about 270 milligrams of stevia.

9. The formulation of claim 2, wherein the formulation comprises per sixteen fluid ounces:
a. about 142 milligrams of potassium sorbate; or
b. about 142 milligrams of sodium benzoate;

10. The formulation of claim 3, wherein the formulation comprises per sixteen fluid ounces:
a. about 600 to about 1200 milligrams of sodium citrate;
b. about 350 to about 750 milligrams of potassium citrate; or
c. about 150 to about 550 milligrams of magnesium citrate.

11. The formulation of claim 3, wherein the formulation comprises per sixteen fluid ounces:
a. about 800 milligrams of sodium citrate;
b. about 450 milligrams of potassium citrate; or
c. about 350 milligrams of magnesium citrate.

12. The formulation of claim 4, wherein the formulation comprises about 15 to about 30 milligrams of cannabidiol per sixteen fluid ounces.

13. The formulation of claim 8, wherein the formulation contains per sixteen fluid ounces:
a. about 142 milligrams of potassium sorbate;
b. about 142 milligrams of sodium benzoate;

14. The formulation of claim 8, wherein the formulation contains per sixteen fluid ounces:
a. about 600 to about 1200 milligrams of sodium citrate;
b. about 350 to about 750 milligrams of potassium citrate; or
c. about 150 to about 550 milligrams of magnesium citrate.

15. The formulation of claim 8, wherein the formulations contains per sixteen fluid ounces:
a. about 800 milligrams of sodium citrate;
b. about 450 milligrams of potassium citrate; or
c. about 350 milligrams of magnesium citrate.

16. The formulation of claim 8, further comprising cannabidiol.

17. The formulation of claim 16, wherein the formulation contains about 15 to about 30 milligrams of cannabidiol per sixteen fluid ounces.

18. A powdered formulation kit for creating a hydrating beverage with the addition of water comprising:
a. about 800 to about 1200 milligrams of calcium lactate;
b. about 950 to about 28000 milligrams of dextrose;
c. about 2000 to about 5000 milligrams of branched-chain amino acids;
d. about 1400 to about 1900 milligrams of citric acid;
e. about 150 to about 1200 milligrams of citrate; and
f. about 270 milligrams of stevia.

19. A powdered formulation kit for creating a hydrating beverage with the addition of water comprising:
a. about 1000 milligrams of calcium lactate;
b. about 10000 milligrams of dextrose;
c. about 4000 milligrams of branched-chain amino acids;
d. about 1700 milligrams of citric acid; and
e. about 270 milligrams of stevia.

20. The formulation of claim 19, further comprising:
a. about 800 milligrams of sodium citrate;
b. about 450 milligrams of potassium citrate; or
c. about 350 milligrams of magnesium citrate.
